(19) United States
(12) Patent Application Publication
(10) Pub. No.: US 2025/0050016 A1
(43) Pub. Date: Feb. 13, 2025

(54) SYSTEM AND METHOD FOR MONITORING AN INFUSION PUMP

(71) Applicant: B. Braun Melsungen AG, Melsungen (DE)

(72) Inventors: Jan Schwarz, Melsungen (DE); Maria Buerger, Malsfeld (DE); Klaus Angersbach, Morschen (DE)

(21) Appl. No.: 18/797,688

(22) Filed: Aug. 8, 2024

(30) Foreign Application Priority Data
Aug. 10, 2023 (EP) 23190915.1

(51) Int. Cl. A61M 5/168; A61M 5/142

(52) U.S. Cl. CPC A61M 5/16859; A61M 2005/14208; A61M 2205/33

(57) ABSTRACT

A system for monitoring an infusion pump includes an infusion pump with at least one sensor for acquiring sensor data, a monitoring server with a monitoring module with a receiving unit, and a transmission unit for transmitting the sensor data to the receiving unit of the monitoring module. The monitoring module is configured to determine the state of the infusion pump based on the sensor data and derive a safety-relevant classification of the infusion pump from the state of the infusion pump.

### CROSS-REFERENCE TO RELATED APPLICATION

This application claims priority under 35 U.S.C. § 119 to European Application No. 23190915.1, filed on Aug. 10, 2023, the content of which is incorporated by reference herein in its entirety.

### FIELD

The present disclosure relates to a system and a method for monitoring an infusion pump.

### BACKGROUND

Infusion pumps are medical devices that enable the targeted and automatic administration of fluids, such as medication or nutrients. The safety and correct functioning of these pumps is of crucial importance, as malfunctions can have serious consequences for patients.

Various safety-related tests are currently being carried out to ensure the safety of the infusion pumps. These tests are designed to ensure that the pumps function properly, both during production and throughout the life cycle of the device. Known technical safety checks (TSC) focus on the detection of electrical faults and mechanical defects in the infusion pump and include safety-related work.

The technical safety check is carried out by a specially trained service technician to maintain the appliance and keep it in good working order. The maintenance process is complicated and can be time-consuming. During these tests, the infusion pumps are put into maintenance mode so that they are not available for regular hospital use.

WO 2021/243068 A1 describes a system that enables components to be monitored via sensors in conjunction with a server and, in the event of a malfunction, sends a message to the device in question and displays a maintenance instruction on the device.

The disadvantage of known technical safety tests is that they require the infusion pump to be taken out of operation and the intervention of a special technician or only support the maintenance or repair of the infusion pump after a malfunction has been detected.

### SUMMARY

The present disclosure is therefore based on the object of specifying a system and method that simplifies and automates technical security checks. Furthermore, a computer product and a training data set are to be specified.

With regard to the system, this task is solved according to the present disclosure with a system for monitoring an infusion pump. The system comprises an infusion pump with at least one sensor for acquiring sensor data, a monitoring server with a monitoring module with a receiver unit and a transmission unit for transmitting the sensor data to the receiver unit of the monitoring module, wherein the monitoring module is configured to determine the state of the infusion pump on the basis of the sensor data and to derive a safety-relevant classification of the infusion pump therefrom.

The present disclosure is based on the consideration that an infusion pump should be available without any interruptions in medical operation. Due to the necessary technical safety monitoring or safety checks by a service technician, which require the pump to be taken out of service, it is not available during this time, so that replacement devices must be organized, and organizational measures must be taken.

As has now been recognized, technical safety checks by a service technician can be avoided by automating the technical safety checks by assessing the condition of the infusion pump based on sensor data. This is particularly possible because sensors that are already present in the infusion pump can be used for this purpose. If this data fulfills predetermined or learned conditions, in particular if a selection or all sensor data is within predetermined tolerances, a safety check can be considered fulfilled, so that the manual check can be omitted, and the infusion pump can remain in operation.

The sensor data is integrated to create a safety-relevant classification. The sensor data can be used to predict a degradation of the infusion pump. The sensor data is then preferably integrated in a further step to create the safety-relevant classification. The safety-relevant classification preferably includes the decision as to whether the device is still considered safe and has passed the safety check.

The safety-relevant classification is therefore preferably a different or further classification of the data. For example, the overall system or the infusion pump can still be considered safe, even though a predictive score would already require maintenance. The other case is also conceivable, namely that the predictive score is not yet reached, but the overall system or infusion pump is already considered unsafe in terms of the safety-relevant classification.

The system therefore not only carries out predictive maintenance of the infusion pump, but also a safety-relevant classification as part of a safety check (STK) and helps the user to run through this automatically and without any intervention on their part.

Advantageously, the safety-relevant classification includes the prediction of a failure and/or the proper functioning of the infusion pump over the running time.

The safety-relevant classification is preferably carried out in cycles, whereby a safety-relevant monitoring of the infusion pump is deemed to have been passed if the infusion pump has functioned properly within the last cycle. In this case, a safety-relevant manual test by a service technician can be omitted so that the infusion pump is still available for its operating status.

The transmission unit preferably transmits the recorded sensor data to the receiver unit essentially continuously or at regular intervals. Depending on the data gap, interpolations (linear or spline) are preferably carried out and the missing data values are approximated In the case of larger data gaps, however, the trend analysis is advantageously aborted and a reinitialization is carried out. Conversely, this means that the previously used data is declared invalid, and the trend analysis cannot be performed.

In a preferred embodiment, the monitoring module comprises a customizable and/or learnable model for safety-related classification. In this way, the model can be adapted to the specific technical situation, i.e. the specific pump and the specific sensors, and criteria for proper functioning of the infusion pump can be adapted over time to any newly acquired knowledge.

The algorithm of the model is advantageously a prediction approach, in particular comprising at least one linear filter model and/or a statistical model, in particular comprising at least one neural network and/or a Gaussian mixture model.

In particular, the model may comprise different models for different groups of sensor data. In a preferred embodiment, therefore, a plurality of models is provided, each model being used specifically for a selection of the total sensor data. For example, certain sensor data may be binary, with one sensor data value leading directly to the classification of failure or malfunction. Other sensor data or their combinations can lead to more complex classifications with intermediate levels.

It is advantageous that there are different models for each sensor, which are not initially correlated. The correlation is only achieved by the fact that in the event of a failure, i.e. defect, the infusion pump is generally marked as defective or, in the event of a “correctable” error, such as display calibration, a calibration is started/initiated by the user.

The monitoring module is preferably configured to set the parameters of the model in an adaptation and/or learning phase. This means that the parameters of the model are set using a training data set. The training can take place in cycles. Advantageously, in the case of a neural network, the network is trained by supervised learning using the training data set. In a preferred embodiment, the learning success of the neural network is then tested using a test data set. If predefined test criteria are not met, the neural network can be trained with modified training parameters (e.g. learning rate) and/or a modified training data set. This can also be applied analogously to the Gaussian mixture model. Here too, the learning success is checked with other data.

In the case of a syringe pump, the monitoring module preferably records and analyzes (i.e. evaluates with the help of the model) sensor data from the group:
claw angle, correlated with the syringe used and its length/diameter as a function of time;
syringe length/diameter of the syringe used as a function of time;
pressure values during insertion of the syringe and/or during the infusion.

In the case of a volumetric pump/peristaltic pump, the monitoring module preferably records and analyzes (i.e. evaluates with the help of the model) sensor data from the group:
movement of the door position sensors by checking the resistance values over time and correlating the values with the motor steps;
color detection of the inserted hose, in particular by means of its clamp, whereby the measured color value is evaluated with the confirmation/change by the user;
acquiring pressure sensor values over time to check whether deviations/changes occur over the course of the service life.

In the case of a volumetric pump/peristaltic pump with a display or syringe pump with a display, the monitoring module preferably records and analyzes (i.e. evaluates with the help of the model) sensor data from the group:
touch sensor values at defined positions on the display, e.g. “Start infusion” button or in input fields as a function of time;
exchange of battery information by a device model, such as the state of charge as a function of time, the capacity of the battery, the number of charging cycles.

The system preferably comprises a transmission device for transmitting information from the monitoring module to the infusion pump. Advantageously, the receiving unit and the transmission device are integrated or combined, i.e. the same unit receives and transmits.

The monitoring module is preferably designed to monitor whether the sensor data of at least one sensor is within a predetermined and/or learned nominal range, whereby the monitoring module is designed to send instructions to the infusion pump with the aid of the transmission device and/or to initiate a maintenance program, preferably a display calibration, of the infusion pump. This is preferably done with sensor data on the properties of the infusion pump, which can be changed in a maintenance step by medical personnel, particularly at short notice. For example, the touch screen or touch display can be recalibrated.

The instructions and/or steps of the maintenance program are preferably shown on a display of the infusion pump. Additionally or alternatively, acoustic signals or voice messages can also be used.

The transmission unit and the receiver unit are preferably designed for wired or wireless communication, in particular with radio, mobile radio, Wi-Fi, Bluetooth. In this way, no additional cables or lines need to be laid, which makes the working area of the infusion pump clearer.

Advantageously, the system further comprises a health data server, which is designed to receive data from the monitoring module and/or to transmit data to the monitoring module. The server preferably receives the data continuously, i.e. whenever there is a change. For example, pressure sensor values are continuously transmitted and monitored during ongoing therapy/promotion.

However, when inserting a tip into the syringe pump, the claw angle and its diameter are primarily measured during the insertion process. Thereafter, the measurement and reporting takes place at irregular intervals (e.g. at 50 ms to 5 s intervals), but always when a new syringe is inserted. The same applies to the air sensor of the peristaltic pump. Here, the data is measured continuously when a disposable is inserted. If possible, the data is bundled and transferred to the server. On the one hand, this is event-controlled, e.g. when a flow rate changes, a tip/tube is inserted or changed, and in any case preferably every 5 seconds.

The health data server is advantageously designed as a cloud server.

The monitoring server advantageously comprises a trend analysis module, which is designed to carry out a trend analysis based on transmitted sensor data. The trend analysis module is preferably designed in the monitoring server in terms of software and/or hardware.

Based on the trend analysis, the trend analysis module preferably carries out a lifetime assessment of the infusion pump. The trend analysis allows faults to be detected and reacted to at an early stage. Preferably, the technician is notified of a precautionary maintenance and, if necessary, a message is sent to the pump to send it for service. However, the latter depends on whether the pump is still being operated on a patient or whether it is unused. In the latter case, it is conceivable to provide the pump with a service notice. In the case of a touch display, it is conceivable in a preferred embodiment to send a “Please recalibrate” field that is operated accordingly by the caregiver. This only takes place outside of ongoing therapy. In particular, creeping prediction can be used here to recognize early on that an error is worsening over time or to adjust the expected runtime/lifetime of the infusion pump.

As an example, it can be recognized in a trend that the touch display requires recalibration. This can be recognized as a trend, for example, by the fact that the pixel positions corresponding to the user's touch gestures for a button whose position is known are constantly changing. For example, the user has to press further and further up on the display to activate the button.

With respect to the method, the above object is solved according to the present disclosure by a method comprising the steps of: acquiring sensor data of an infusion pump; inputting the sensor data into a mathematical model; classifying the state of the infusion pump by the mathematical model. The method is computer-implemented.

In a preferred embodiment, the method comprises an adaptation and/or training step for the mathematical model, in which the model is adapted and/or trained using a training data set.

The training procedure of the statistical models preferably only contains sensor data that has led to a failure or belongs to a fault-free state. It is known a priori whether it is a good case (fault-free) or a bad case (failure). The mathematical/statistical models are trained accordingly. An adaptation phase can also be provided in which the statistical models are adapted and improved using further data (measured values) from the field. This is advantageous if the sensor data is subject to a higher variance. This variance can be incorporated into the learned model through retraining, i.e. adaptation, and thus lead to greater robustness. Adaptation to reality is still possible, i.e. the model is continuously improved/adapted with real data.

The training data set advantageously comprises data from at least one sensor and an associated classification. Advantageously, more than one sensor is included in the considerations. The sensor values are uncorrelated and must therefore be considered separately. Good and bad cases are required in the training data to enable classification

The mathematical model advantageously comprises at least one model from the group: linear filter model and/or a statistical model, in particular comprising at least one neural network and/or a Gaussian mixture model.

The method preferably comprises a method step of monitoring the sensor data of at least one sensor, wherein maintenance instructions are shown on a display of the infusion pump if the sensor data leave a predetermined and/or learned nominal range.

Maintenance instructions essentially refer to instructions for recalibrating the display or touch calibration. In the event of another defect, the corresponding maintenance instruction can be an indication that the pump should be handed over to the service technician (because there is a defect).

With respect to the computer product, the above problem is solved according to the present disclosure by a computer program product comprising instructions which, when executed on a computer, perform a method described above.

With regard to the training data set for training a neural network in a method with a training step described above, the above task is solved according to the present disclosure with a training data set which has the following structure. The training data set preferably contains only sensor data which have both led to a failure or belong to a fault-free state. It is known a priori whether it is a good case (fault-free) or a bad case (failure). The mathematical/statistical models are trained accordingly.

In an adaptation phase, the statistical models can be adapted and improved using additional data (measured values) from the field. This is particularly advantageous if the sensor data is exposed to a higher variance. This variance can be incorporated into the learned model through retraining, i.e. adaptation, and thus lead to greater robustness. To a certain extent, the training data set comprises the a priori knowledge about defective and error-free sensor data. In addition, the sensor data of a different number of infusion pumps (peristaltic or syringe pumps) are preferably used for training to cover the scattering of the sensors due to component tolerances. Furthermore, the sensor data of infusion pumps from different batches and of different ages are advantageously used to better/more accurately map the scattering of the sensor data.

The present disclosure also relates to a system for monitoring an ambulatory pump or a dialysis machine, comprising an ambulatory pump or dialysis machine with at least one sensor for acquiring sensor data, a monitoring server with a monitoring module with a receiving unit, a transmission unit for transmitting the sensor data to the receiving unit of the monitoring module, wherein the monitoring module is configured to determine the state of the ambulatory pump/dialysis machine on the basis of the sensor data and to derive a safety-relevant classification of the ambulatory pump/dialysis machine therefrom.

The preferred embodiments described above for the system for monitoring an infusion pump in the description are also preferred embodiments for this system for monitoring an ambulatory pump or a dialysis machine, provided that they are technically directly or equivalently transferable for the person skilled in the art.

The advantages of the present disclosure lie in particular in the fact that an automatic mechanism is provided to detect failures at an early stage and/or to extend the maintenance of the device based on trend analyses based on sensor values. In this way, the infusion pump can remain in operation for as long as possible. By monitoring the infusion pump and/or performing trend analyses, maintenance intervals can be postponed or brought forward, thus ensuring the functional use of the infusion pump. As maintenance generally has to be carried out every two years in Germany, continuous monitoring makes it possible to carry out the inspection based on the data available. This saves time and money.

### BRIEF DESCRIPTION OF THE DRAWINGS

An embodiment of the present disclosure is explained in more detail with the aid of a drawing and schematized representation.

FIG. 1 shows a system in a preferred embodiment with an infusion pump, a monitoring server and a health data server;

FIG. 2 shows a system in a further preferred embodiment with an infusion pump, a monitoring server and a health data server;

FIG. 3 shows a display of an infusion pump in a first state;

FIG. 4 shows the display according to FIG. 3 in a second state;

FIG. 5 shows the display according to FIG. 3 in a third state; and

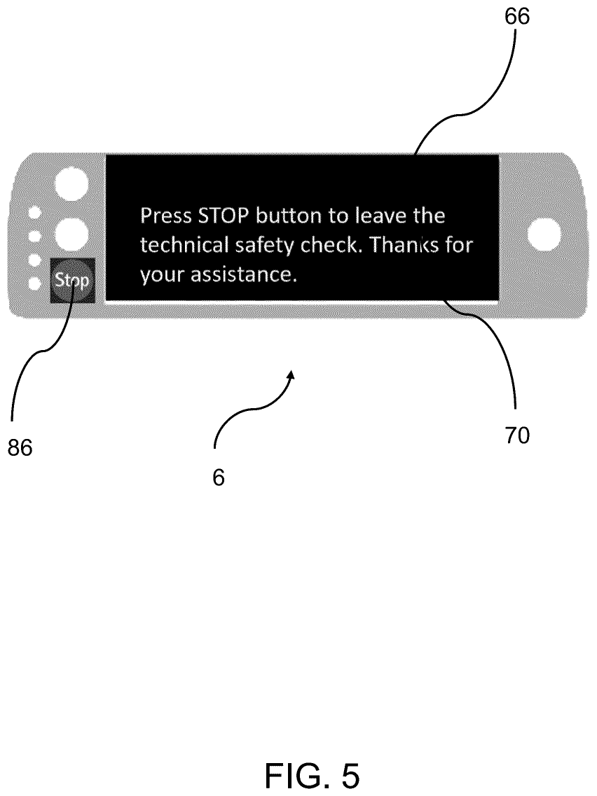

FIG. 6 shows a flow chart of a method in a preferred embodiment.

Identical parts are marked with the same reference signs in all figures.

### DETAILED DESCRIPTION

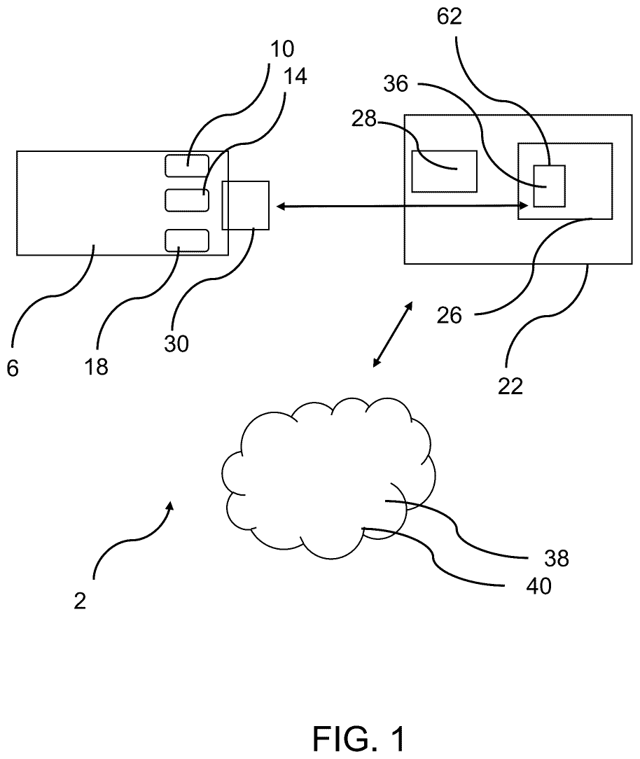

A system 2 shown in FIG. 1 comprises an infusion pump 6 with a plurality of sensors 10, 14, 18. The system 2 comprises a monitoring server 22 with a monitoring module 26 which is implemented in hardware and/or software in the monitoring server 22. The system 2 further comprises a transmission unit 30 which is connected to the sensors 10, 14, 18 or a processor to which the sensors are connected and which is configured or set up to transmit the data or sensor data acquired by the sensors to a receiving unit 36 of the monitoring module 26.

The system 2 further comprises a health data server 38, which in the present case is preferably designed as a cloud server 40, wherein the monitoring server 22 and the cloud server 40 are set up for data transmission from the monitoring server 22 to the cloud server and/or from the cloud server 40 to the monitoring server 22.

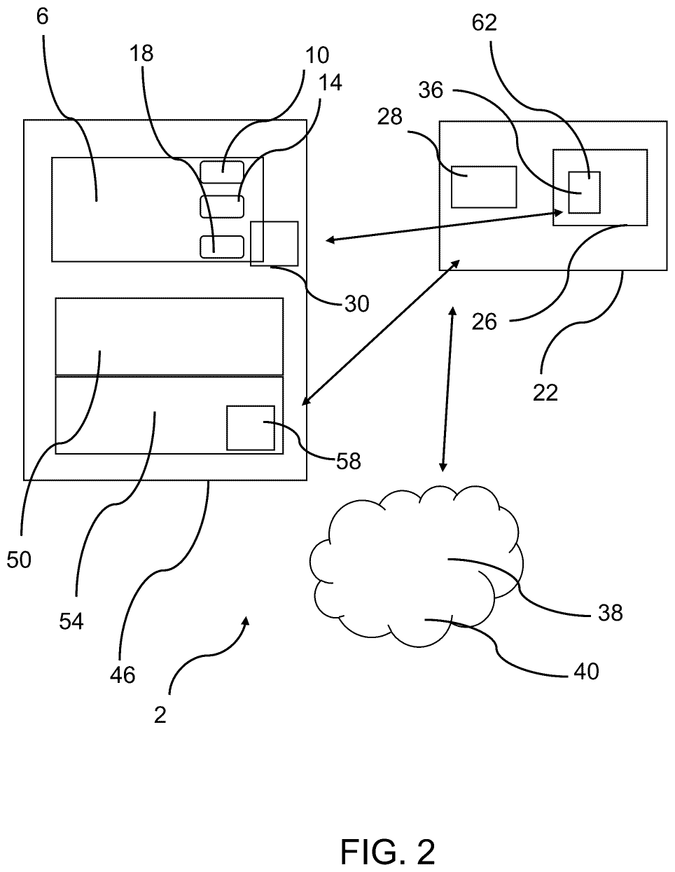

In FIG. 2, a system 2 is shown in another preferred embodiment. The system 2 comprises an infusion workstation 46, which comprises the infusion pump 6 as well as a rack system 50 and a communication system 54. In this embodiment, the infusion workstation 46 comprises a transmission unit 30. The communication system 54 may include a transmission unit 58 for exchanging data with the monitoring server 22. The infusion workstation 46 is designed to accommodate a plurality of infusion pumps 6, in particular up to 24 infusion pumps, in the rack system 50, which forms an accommodation system for the infusion pumps 6. In this configuration, a common transmission unit 30 is thus provided for all infusion pumps 6 arranged in the rack system 50.

Each pump preferably sends its data individually to the monitoring server 22 via its communication unit. Each pump has a unique identification, which allows it to be assigned accordingly.

The two systems 2 shown in FIGS. 1 and 2 each have a transmission device 62 for transmitting information from the monitoring module 26 to the infusion pump 6. In the present case, the receiving unit 36 and the transmission device 62 are integrated. The monitoring server 22 of each of the systems 2 also comprises a trend analysis module 28.

The sensors 10, 14, 18 can be sensors from the group:
sensor for measuring the claw angle
sensor for measuring the spray diameter
pressure sensor for measuring the pressure in the syringe
door sensor of a volumetric pump or peristaltic pump
color detection sensor for a peristaltic pump
touch display sensor
sensor for battery charge status
terminal on the appliance side of the peristaltic pump (for monitoring whether the hose is disconnected, e.g. when the door is opened)
sensor for checking an inserted hose clamp on the peristaltic pump
connection to mains voltage (yes/no)
position of the plunger blade on a syringe pump

Preferably, the nurse call is monitored, i.e. it is monitored whether the signal is actually present at the connector. Preferably, the LEDs (red, yellow, white) are monitored. For this purpose, the voltage values are checked for plausibility/drift and fed to a model accordingly.

The monitoring module 26 in the embodiments shown in FIGS. 1 and 2 is adapted to perform a classification of the condition of the infusion pump 6 based on the incoming sensor data from the sensors 10, 14, 18. The monitoring module 26 is designed to be adapted or trained to perform said classification of the condition.

In embodiments in which the model is a statistical model, in particular a neural network, a training data set is provided which contains sensor data. Furthermore, a test data set is provided to test the quality or accuracy of the trained model.

Supervised learning is preferred to ensure that the model is trained correctly. Reinforced learning can also be used provided that the underlying Markov chain is understandable/traceable, i.e. that the Markov model is validated with regard to the circumstances. An exemplary training data set contains the measured values for syringe size recognition. When the syringe is inserted into a syringe pump, the diameter is measured using resistance values. These resistance values vary on the one hand depending on the syringe size (e.g. a 50 ml syringe has a larger diameter than a 20 ml syringe) and on the other hand depending on the insertion. It is also known what the nominal diameter of the tip is and whether it is a good or bad sample.

A similar example can be determined for the door position. An exemplary system has a total of 3 different door positions (open, closed and “park position”). Due to component tolerances, these measured values also vary by a nominal value for these positions. Here too, threshold values can be used to generate good and bad patterns that are used for training.

Furthermore, the touch positions on the display or screen can be selected relatively easily as an input variable. The training data set knows the positions for individual graphical elements on the screen as well as a variance of the data points to a nominal value. If the measured value is within this range, it is considered good. If a measured value lies outside a range, it is marked as incorrect.

One of the main tasks of the current technical safety check is to check whether buttons react to being touched and whether the infusion pump's alarm indicators (e.g. yellow status light, red status light, green infusion status light) are working properly. Currently, devices must undergo a safety check every 2 years. The present disclosure enables individual checks to be outsourced to specialist personnel (e.g. checking LEDs or testing touchscreens) to save time and money. A second aspect of the present disclosure relates to the fact that continuous monitoring of the device and its measured values makes it possible to recognize very clearly whether a fault is creeping in or not. This makes it possible to automate this part and thus avoid the need for a dedicated safety check, because the data is checked continuously (or at shorter intervals than a safety check would provide).

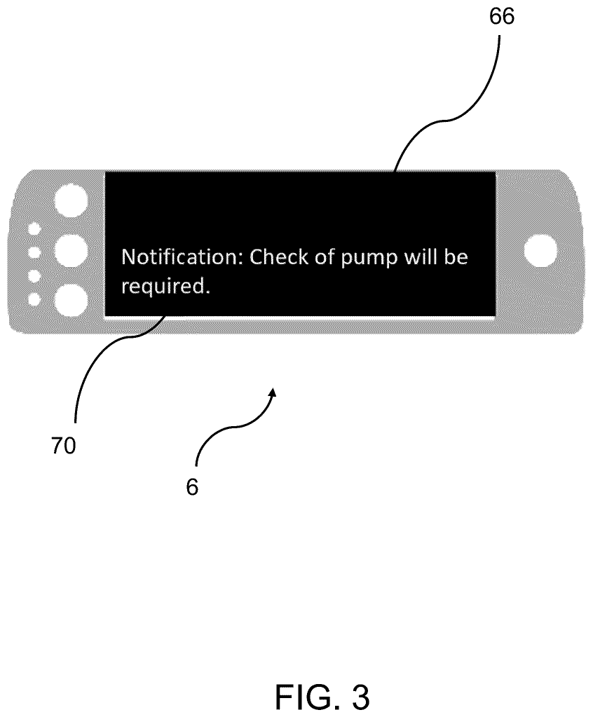

One aspect of the present disclosure is therefore to send information messages to the infusion pump 6 through the monitoring server 22, thereby informing the user or medical professional. In the present case, as shown in FIG. 3, the medical staff is informed by a message 70 on a display 66 of the infusion pump 6, which is designed as a touch display, that a message for checking the status lights will be displayed from the next day for the pumps that are not infusing. This message is only displayed when the infusion pump is inactive.

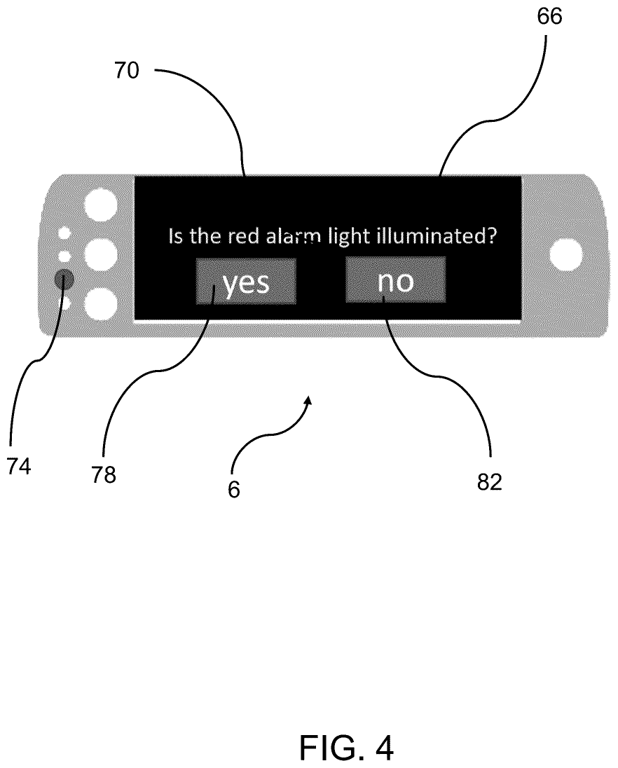

Regardless of whether such a message is posted or not, the monitoring server 22 will display a message 70 on the display 66 of the infusion pump 6 that the user should check the status lights, as exemplified in FIG. 4 for a red alarm light 74.

The status lights are activated one after the other and the user must confirm that the respective status light has lit up. A confirmation is carried out on the touchscreen to check the reaction of the display 66 to touch inputs. For this purpose, two buttons 78, 82 are shown on the display, with one button 78 representing the response “Yes” and the other button representing the response “No”.

After the respective status light has been displayed and confirmed, the user receives a short message to press a hard key (i.e. not a button on the touch display, but a mechanical button) to exit the dialog. By using the touch screen and hard keys to confirm the instructions, the mandatory functionalities of the user interface are also checked. A dialog described above with the user through displayed instructions and button presses can also be supplemented by acoustic signals to check the loudspeaker and/or a backup alarm.

Finally, once all tests have been completed, the technical safety check is ended and the user is shown a corresponding message 70 on the display 66, which he can acknowledge by pressing a stop button 86, which is designed as a hard key in the present case, thereby ending the safety check.

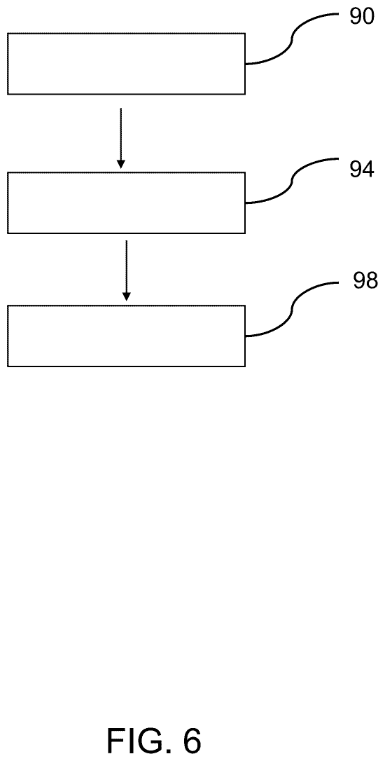

In FIG. 6, a method for monitoring an infusion pump 6 is shown in a preferred embodiment. In a first method step 90, sensor data of an infusion pump 6 are recorded, which are measured by sensors. The sensor data is recorded continuously or at regular intervals. In a second step 94, which is carried out at least partially in parallel with the first step 90, the sensor data is entered into a mathematical model; this is done automatically and at regular intervals. The model is a linear filter model and/or a statistical model, in particular comprising at least one neural network and/or a Gaussian mixture model.

In a third process step 98, which runs at least partially parallel to process step 94, the status, in particular the safety-relevant status, of the infusion pump 6 is classified. The specific classification can be designed differently depending on the model. In simple linear models, for example, the classification can represent a binary decision: Condition is OK from a safety point of view/Condition is not OK; the infusion pump must be serviced or repaired.

The sensor data is measured and compared with the model. This means that, based on the sensor value and the previously measured values, an assessment/prediction (via filter process or machine learning) of a “device defective” or “device ready for operation” status is obtained.

If the device is marked as “Device ready for operation”, nothing happens. The device can still be used. However, if the status “Device defective” is estimated, this is displayed in the form of a message on the device (in the form of an error message). In addition, a message to the service technician is created on the server. It is also possible to send emails or other messages via the server to the service technician for the purpose of repairing the infusion pump.

### LIST OF REFERENCE SIGNS

2 system
6 infusion pump
10 sensor
14 sensor
18 sensor
22 monitoring server
26 monitoring module
28 trend analysis module
30 transmission unit
36 receiving unit
38 health data server
40 cloud server
46 infusion workstation
50 rack system
54 communication system
58 transmission unit
62 transmission device
66 display
70 message
74 red alarm light
78 button
82 button
86 stop button
90 step
94 step
98 step

1. A system for monitoring an infusion pump, the system comprising:
an infusion pump with at least one sensor for acquiring sensor data;
a monitoring server with a monitoring module with a receiving unit; and
a transmission unit for transmitting the sensor data to the receiving unit of the monitoring module,
the monitoring module being configured to determine a state of the infusion pump based on the sensor data and to derive a safety-relevant classification of the infusion pump from the state of the infusion pump.

2. The system according to claim 1, wherein the safety-relevant classification comprises a prediction of a failure and/or of a proper functioning of the infusion pump over a running time.

3. The system according to claim 2, wherein the safety-relevant classification takes place in cycles, and wherein a safety-relevant monitoring of the infusion pump is deemed to have been passed when the infusion pump has functioned properly within a cycle.

4. The system according to claim 1, wherein the transmission unit transmits the sensor data to the receiving unit substantially continuously or at regular time intervals.

5. The system according to claim 1, wherein the monitoring module comprises a model that is customizable and/or learnable for the safety-relevant classification. The system according to claim 5, wherein the model comprises an algorithm that is a prediction approach.

7. The system according to claim 1, wherein the monitoring module acquires and analyzes sensor data in a syringe pump from:
a claw angle, correlated with a syringe used and a length/diameter of the syringe as a function of time;
a syringe length/diameter of the syringe used as a function of time; and
pressure values during insertion of the syringe and/or during an infusion.

8. The system according to claim 1, wherein the monitoring module acquires and analyzes sensor data in a volumetric pump/peristaltic pump from:
movement of door position sensors by checking resistance values over time and correlating the resistance values with motor steps;
color detection of an inserted hose, whereby a measured color value is evaluated with a confirmation/change by a user; and
acquiring pressure sensor values over time to check whether deviations/changes occur during a service life.

9. The system according to claim 1, wherein the monitoring module acquires and analyzes sensor data in a volumetric pump/peristaltic pump with a display or syringe pump with a display from:
touch sensor values at defined positions on the display; and
exchange of battery information by a device model.

10. The system according to claim 1, further comprising a transmission device for transmitting information from the monitoring module to the infusion pump.

11. The system according to claim 10, wherein the monitoring module is designed to monitor whether the sensor data of at least one sensor is within a predetermined and/or learned nominal range, and to send instructions to the infusion pump and/or to initiate a maintenance program of the infusion pump with assistance from the transmission device.

12. The system according to claim 11, wherein the instructions and/or steps of the maintenance program are shown on a display of the infusion pump.

13. The system according to claim 1, further comprising a health data server configured to receive data from the monitoring module and/or to transmit data to the monitoring module.

14. The system according to claim 1, wherein the monitoring server comprises a trend analysis module, which is designed to perform a trend analysis based on transmitted sensor data.

15. The system according to claim 14, wherein the trend analysis module performs a lifetime estimation of the infusion pump based on the trend analysis.

16. A method for monitoring an infusion pump comprising the steps of:
acquiring sensor data from the infusion pump;
entering the sensor data into a mathematical model;
classifying a state of the infusion pump using the mathematical model.

17. The method according to claim 16, further comprising an adaptation and/or training step for the mathematical model, in which the mathematical model is adapted and/or trained with assistance from a training data set.

18. The method according to claim 17, wherein the training data set comprises data of at least one sensor and an associated classification.

19. The method according to claim 16, further comprising the step of monitoring data of at least one sensor, wherein maintenance instructions are displayed on a display of the infusion pump when the sensor data leaves a predetermined and/or learned nominal range.

20. A computer program product comprising instructions which, when executed on a computer, perform the method according to claim 16.
